(19) United States
(12) Patent Application Publication
(10) Pub. No.: US 2007/0287843 A1
(43) Pub. Date: Dec. 13, 2007

(54) Methods and Intermediates for the Synthesis of Delta-9 Tetrahydrocannabinol

(76) Inventors: John E. Cabaj, Sheboygan, WI (US); Richard J. Pariza, Zion, IL (US); Julie M Lukesh, Huron, WI (US)

Correspondence Address: Quarles & Brady, 411 E. Wisconsin Avenue, Milwaukee, WI, 53202, US

(21) Appl. No.: 11/547,892

(22) PCT Filed: Apr. 7, 2005
(86) PCT No.: PCT/US05/11974

§ 371 Date: Aug. 2, 2007

Related U.S. Application Data
(60) Provisional application No. 60/560,327, filed on Apr. 7, 2004.
(60) Provisional application No. 60/607,080, filed on Sep. 3, 2004.

(51) Int. Cl. C07D 311/80; C07C 35/18

(52) U.S. Cl. 549/390; 568/823

(57) ABSTRACT

Processes are disclosed for the synthesis of Delta-9 tetrahydrocannabinol which result in an improved Y-THC/Y-THC ratio, and intermediates are disclosed that may be used in the synthesis of Delta-9 tetrahydrocannabinol such that improved Y-THCIY-THC ratios are achieved. The intermediates may be cyclic compounds prepared from 2-Carene. There is also provided a scaleable process for the preparation of (+)-p-menth-2-ene-1,8-diol, another intermediate used in the synthesis of delta-9-tetrahydrocannibinol.

### CROSS-REFERENCES TO RELATED APPLICATIONS

This application claims priority from U.S. Provisional Patent Application No. 60/560,327 filed Apr. 7, 2004 and U.S. Provisional Patent Application No. 60/607,080 filed Sep. 3, 2004.

### STATEMENT REGARDING FEDERALLY SPONSORED RESEARCH

Not Applicable.

### BACKGROUND OF THE INVENTION

1. Field of the Invention

The present invention relates to processes for the synthesis of Delta-9 tetrahydrocannabinol, and more particularly to intermediates used in the synthesis of Delta-9 tetrahydrocannabinol.

2. Description of the Related Art

Delta-9 tetrahydrocannabinol (Δ⁹-THC), the active ingredient in marijuana, is a tricyclic terpene currently being used for appetite stimulation in cancer and AIDS patients. Various methods for synthesizing Δ⁹-THC are known and in one method, (+)-p-Menth-2-ene-1,8-diol 1 is reacted with olivetol 2 to prepare delta-9-tetrahydrocannibinol 3. See FIG. 1. See, for example, Razdan, Tetrahedron Lett., 1979, p. 681; Stoss, Synlett, 1991, p. 553; U.S. Pat. No. 5,227,537; and PCT International Publication Nos. WO 02/096899 and WO 02/096846. These documents and all other documents cited herein are incorporated herein by reference.

In order to develop a scaleable synthesis of delta-9-tetrahydrocannibinol 3 using these methods, one needs to produce significant quantities of the (+)-p-Menth-2-ene-1,8-diol 1 intermediate. In one known method, (+)-p-Menth-2-ene-1,8-diol 1 can be prepared from (+)-trans-2,3-epoxy-cis-carane (2-carene epoxide) 5a using the method of Prasad as shown in FIG. 2 and as described at Tetrahedron, 1976, p. 1437. However, the yields using this method can be low. In another method described in U.S. Pat. No. 3,814,733 for preparing the (+)-p-Menth-2-ene-1,8-diol 1 intermediate, the treatment of 2-carene epoxide 5a with sulfuric acid in water gives a 50% yield of (+)-p-Menth-2-ene-1,8-diol. Yet another method for preparing (+)-p-Menth-2-ene-1,8-diol 1 has been reported in WO 02/096846 wherein the method involves stirring the 2-carene epoxide 5a in pH 5.7 to 5.9 water at 40° C. without a catalyst. It is reported that (+)-p-Menth-2-ene-1,8-diol 1 can be obtained in 82% yield using these conditions after exhaustive extraction (seven extractions) with ethyl acetate followed by concentration to dryness.

It is further reported in WO 02/096846 that a 40/60 mixture of 2-carene/3-carene can be used to produce (+)-p-Menth-2-ene-1,8-diol 1 without the need to separate the two regioisomers. Thus, epoxidation of a 42/58 mixture of 2-carene/3-carene using catalytic methyl trioxorhenium and hydrogen peroxide as the stoichiometric oxidant presumably gives a mixture of 2-carene epoxide 5a and 3-carene epoxide 5b. See FIG. 3. This mixture is then treated with pH 5.8 water at 30° C. After ethyl acetate extraction and concentration to dryness, the (+)-p-Menth-2-ene-1,8-diol 1 is isolated in an overall yield of 35% based on the amount of contained 2-carene in the 40/60 mixture.

Because of the difficulties and/or low yields of these known methods, there exists a need for a more straightforward, scaleable method for preparing (+)-p-Menth-2-ene-1,8-diol 1 from 2-carene epoxide 5a.

The general synthetic approach to 2-THC 3 as shown in FIG. 1 involves the functionalization of (+)-p-Menth-2-ene-1,8-diol 1 which is reacted with olivetol 2 to give Δ⁹-THC 3 and Δ⁸-THC 4. The relative stereochemistry at carbons 6a and 10a is controlled by the single stereogenic center present in (+)-p-Menth-2-ene-1,8-diol 1.

One major problem with the approach of FIG. 1 is that it is low yielding and the purification is tedious. For instance, it is reported in WO 02/096899 and WO 02/096846 that the chemical synthesis and the isolation of Δ⁹-THC 3 are both challenging because Δ⁹-THC 3 has a very high boiling point, Δ⁹-THC 3 is prone to acid-catalyzed isomerization to the thermodynamically more stable Δ⁸ isomer 4, Δ⁹-THC 3 is easily oxidized by oxygen to inactive cannabinol, and Δ⁹-THC 3 is sensitive to light and heat. In particular, the separation of Δ⁸-THC 4 from Δ⁹-THC 3 is exceedingly difficult by conventional means. Thus, synthetic approaches which maximize the Δ⁹-THC/Δ⁸-THC ratio would be advantageous.

Therefore, there is also a need for processes for the synthesis of Delta-9 tetrahydrocannabinol which result in an improved Δ⁹-THC/Δ⁸-THC ratio. Furthermore, there is a need for intermediates that may be used in the synthesis of Delta-9 tetrahydrocannabinol such that improved Δ⁹-THC/Δ⁸-THC ratios are achieved.

### BRIEF SUMMARY OF THE INVENTION

The foregoing needs are met by the present invention wherein (+)-p-Menth-2-ene-1,8-diol is prepared from 2-carene epoxide. In one version of the process according to the invention, a reaction mixture is prepared including 2-carene epoxide, a solvent in which (+)-p-Menth-2-ene-1,8-diol is insoluble, water, and an acid catalyst. After a time period, (+)-p-Menth-2-ene-1,8-diol precipitates from the reaction mixture. The reaction mixture is then filtered to remove (+)-p-Menth-2-ene-1,8-diol from the reaction mixture.

In another version of the process according to the invention, a reaction mixture is prepared including a mixture of 2-carene epoxide and 3-carene epoxide, a solvent in which (+)-p-Menth-2-ene-1,8-diol is insoluble, water, and an acid catalyst. After a time period, (+)-p-Menth-2-ene-1,8-diol precipitates from the reaction mixture. The reaction mixture is then filtered to remove (+)-p-Menth-2-ene-1,8-diol from the reaction mixture.

The foregoing needs are also met by the present invention in which cyclic compounds prepared from 2-Carene, or cyclic compounds prepared from mixtures of 2-Carene and 3-Carene, are reacted with unsubstituted resorcinol or a substituted resorcinol (such as olivetol) to produce Delta-9 tetrahydrocannabinol with an improved Δ⁹-THC/Δ⁸-THC ratio.

In one form, the cyclic compound prepared from 2-Carene has the following formula: 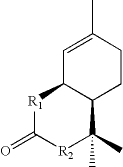
wherein R₁ is selected from O, N and S and R₂ is selected from O, N and S.

In another form, the cyclic compound prepared from 2-Carene has the following formula:

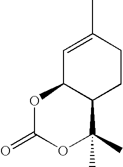

In yet another form, the cyclic compound prepared from 2-Carene has the following formula: 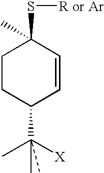
wherein S is sulfur or sulfoxide or sulfone; R is alkyl or cycloalkyl; Ar is aryl; and X is OH, OR, OCOR, OCOAr, O-substituted silyl groups, a halogen, or nothing when the dashed line is present as a double bond with the lowermost carbon. All enantiomers and diastereomers of these compounds are suitable for practicing the invention.

It is therefore an advantage of the present invention to provide an improved method for preparing (+)-p-Menth-2-ene-1,8-diol, an intermediate that may be used in the synthesis of Delta-9 tetrahydrocannabinol.

It is another advantage of the invention to provide alternative intermediates that may be used in the synthesis of Delta-9 tetrahydrocannabinol such that improved Δ⁹-THC/Δ⁸-THC ratios are achieved.

These and other features, aspects, and advantages of the present invention will become better understood upon consideration of the following detailed description, drawings, and appended claims.

### BRIEF DESCRIPTION OF THE DRAWINGS

FIG. 1 is a known scheme for preparing delta-9-tetrahydrocannibinol 3 from (+)-p-Menth-2-ene-1,8-diol 1 and olivetol 2.

FIG. 2 is a known scheme for preparing (+)-p-Menth-2-ene-1,8-diol 1 from 2-carene epoxide 5a.

FIG. 3 is a known scheme for preparing (+)-p-Menth-2-ene-1,8-diol 1 from a mixture of 2-carene epoxide 5a and 3-carene epoxide 5b.

FIG. 4 is a scheme according to the invention for preparing (+)-p-Menth-2-ene-1,8-diol 1 from 2-carene epoxide 5a, which is prepared from 2-carene.

FIG. 5 is a scheme according to the invention for preparing (+)-p-Menth-2-ene-1,8-diol 1 from a mixture of 2-carene epoxide 5a and 3-carene epoxide 5b.

FIG. 6 is a scheme for synthesizing an intermediate 6 according to the invention.

FIG. 7 is a scheme for producing Delta-9 tetrahydrocannabinol 3 and Delta-8 tetrahydrocannabinol 4 from olivetol 2 and the intermediate 6 produced using the scheme of FIG. 6.

FIG. 8 is another scheme for producing diol 1 used in synthesizing an intermediate according to the invention.

FIG. 9 is a scheme for producing an intermediate II according to the invention that may be used in the synthesis of Delta-9 tetrahydrocannabinol.

FIG. 10 is a scheme for producing Delta-9 tetrahydrocannabinol 3 from olivetol 2 and the intermediate II produced using the scheme of FIG. 9.

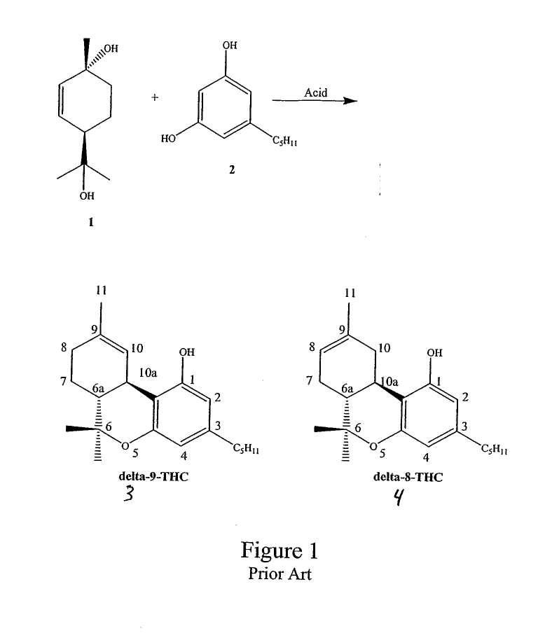

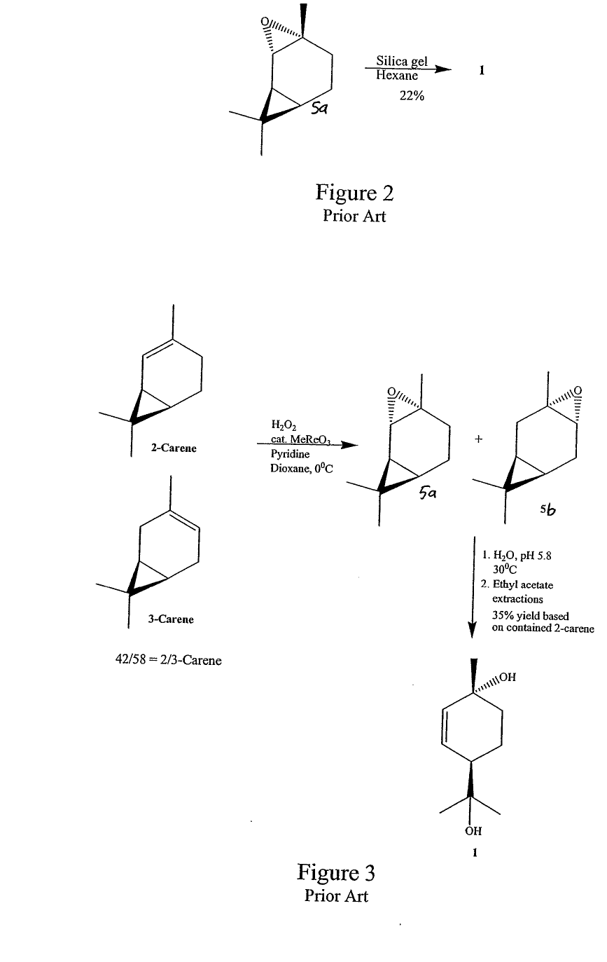

### DETAILED DESCRIPTION

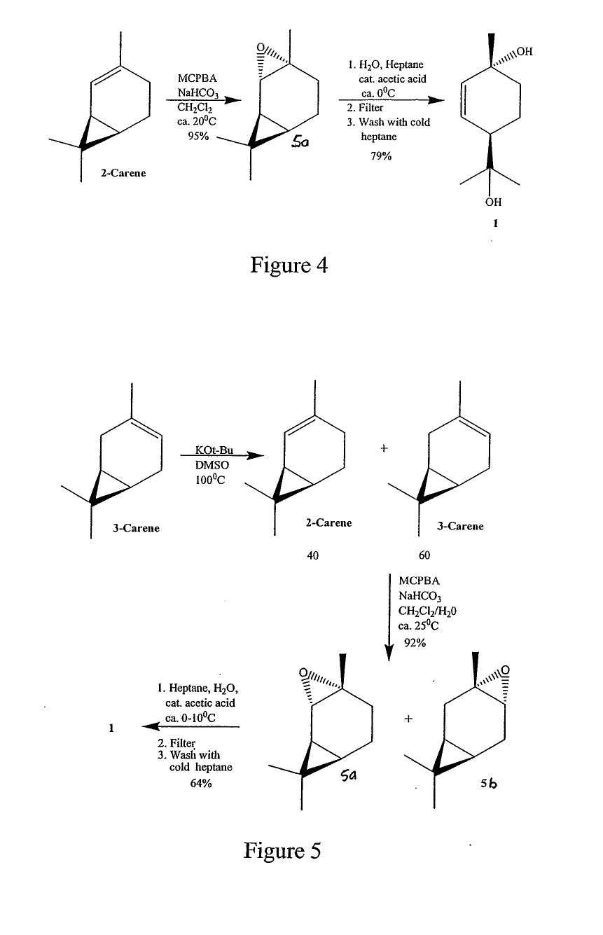

We have discovered that (+)-p-Menth-2-ene-1,8-diol 1 can be produced from (+)-trans-2,3-epoxy-cis-carane (2-carene epoxide) 5a using a much more straightforward, scaleable process as shown in FIG. 4. Treatment of 2-carene with buffered 3-chloroperbenzoic acid (MCPBA) in a biphasic mixture of methylene chloride/water gives 2-carene epoxide 5a in good yield (over 90%) after extractive workup and concentration. Treatment of a heptane solution of 2-carene epoxide 5a with water and catalytic acetic acid results in precipitation of (+)-p-Menth-2-ene-1,8-diol 1 from the reaction mixture. The slurry is simply filtered and washed with cold heptane to give (+)-p-Menth-2-ene-1,8-diol 1 in a yield of at least 70%. Thus, the time and expense of the exhaustive ethyl acetate extractions used in the procedure of WO 02/096846 are avoided using the procedure of the present invention.

In an example embodiment of the invention, 2-carene epoxide is stirred in a solvent in which (+)-p-Menth-2-ene-1,8-diol is insoluble, and water and an acid catalyst are added to the 2-carene epoxide and solvent. Thereafter, (+)-p-Menth-2-ene-1,8-diol precipitates from the mixture. The reaction mixture may then be filtered to remove (+)-p-Menth-2-ene-1,8-diol from the reaction mixture. The (+)-p-Menth-2-ene-1,8-diol may be further washed with the solvent and dried in an oven to yield a solid. Suitable solvents include, but are not limited to, cyclohexane (or other hydrocarbon solvents), methyl-t-butyl ether, diethyl ether, methylene chloride, chloroform, toluene (or other aromatic solvents). Mixed solvents that can be used include, but are not limited to, methyl-t-butyl ether/heptane, methylene chloride/heptane, isopropanol acetate/heptane, and t-butanol/heptane.

The 2-carene epoxide may be prepared using known methods such as the epoxidation of 2-carene with 3-chloroperbenzoic acid. Non-limiting examples of the solvent include C₅-C₁₂ alkanes, and ether solvents such as methyl-t-butyl ether and diethyl ether. In general, any non-nucleophilic organic solvent should be suitable in the process of the invention. The preferred solvent is heptane in that (+)-p-Menth-2-ene-1,8-diol is not soluble in heptane and thus readily precipitates out of solution thus protecting itself from further reaction. It is preferred that the 2-carene/solvent mixture be adjusted to a temperature of 25° C. or below, preferably −5° to 10° C.

Preferably, the catalyst is selected from the group consisting of aliphatic carboxylic acids, aromatic carboxylic acids, sulfonic acids, pyridinium acids, ammonium acids, and mixtures thereof, and the catalyst is soluble in the solvent. Other suitable acid catalysts include, but are not limited to, acetic acid/t-butanol, benzoic acid, formic acid, trifluoroacetic acid, and potassium phosphate monobasic. Most preferably, the catalyst is acetic acid because it is soluble in heptane and easily washed out or removed during the drying of the solid (+)-p-Menth-2-ene-1,8-diol. Regarding the water used in the process, only 1.0 molar equivalents of water is required for each equivalent of 2-carene epoxide.

We have also discovered that a mixture of 2-carene/3-carene can be used to produce (+)-p-Menth-2-ene-1,8-diol 1 as shown in FIG. 5. This process is higher yielding and involves fewer processing steps than known methods. The treatment of 3-carene with potassium t-butoxide in DMSO at 100° C. gives a 40:60 mixture of 2-carene/3-carene (area percent by GC). See Acharya, J. Org. Chem., 1967, 89, 1925. Treatment of this mixture to the same conditions as those which were used for pure 2-carene epoxide 5a gives (+)-p-Menth-2-ene-1,8-diol 1 in moderate to good yield. It should be noted that the filtrate contains unreacted 3-carene epoxide 5b along with other process impurities. The crude diol can be recrystallized from ethyl acetate/heptane (1/2 by volume) to give (+)-p-Menth-2-ene-1,8-diol 1 as a crystalline solid. It is understood that other known methods can potentially be used to form the epoxide mixture. Regarding the water used in this version of the process, only 1.0 molar equivalents of water is required for each equivalent of 2-carene epoxide, which means that if you start with a 40/60 mixture of 2/3 carene epoxide, you should need 0.4 equivalents of water. Extra water is not a problem since the 3-carene epoxide seems to be inert towards water and does not react.

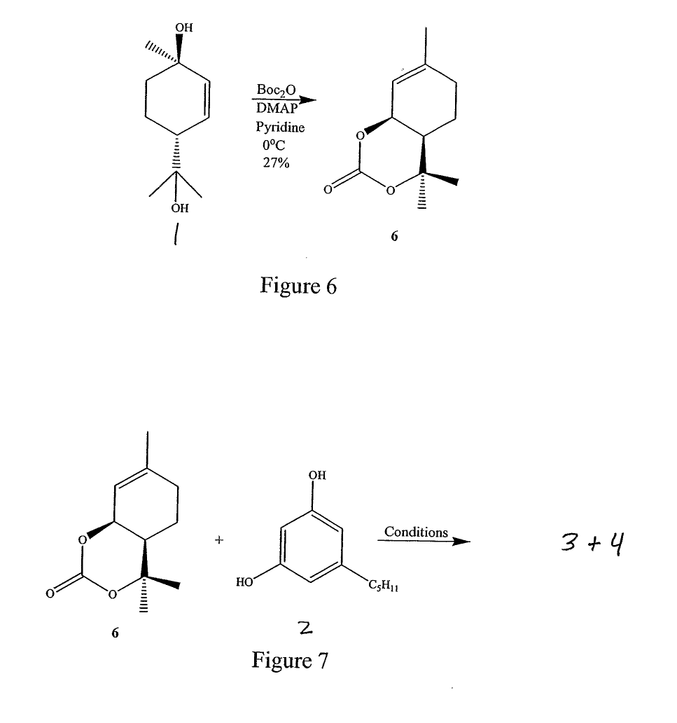

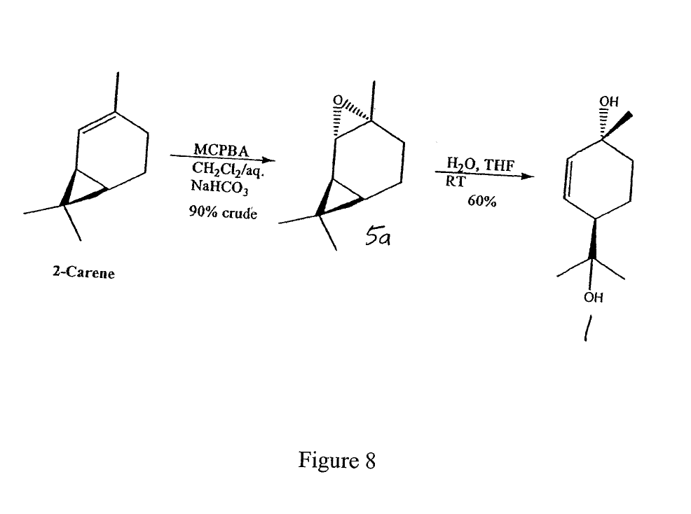

In another version of the invention, a chiral non-racemic carbonate 6 is used as an intermediate in the synthesis of Δ⁹-THC 3. Treatment of diol 1 with di-tert-butyl-dicarbonate in the presence of a catalytic amount of 4-(dimethylamino)-pyridine gives carbonate 6 as a crystalline solid as shown in FIG. 6. The diol 1 may be synthesized as shown in FIG. 8. Optionally, the oxygen heteroatoms in the carbonate may be sulfur or nitrogen.

Reaction of carbonate 6 with olivetol 2 in the presence of various Lewis acids yields Δ⁹-THC 3 and Δ⁸-THC 4 as shown in FIG. 7. The carbonate 6 is a viable intermediate in the synthesis of Δ⁹-THC 3. In particular, reaction of the carbonate 6 with olivetol 2 in the presence of various Lewis acids has been demonstrated as useful in synthesizing Δ⁹-THC 3.

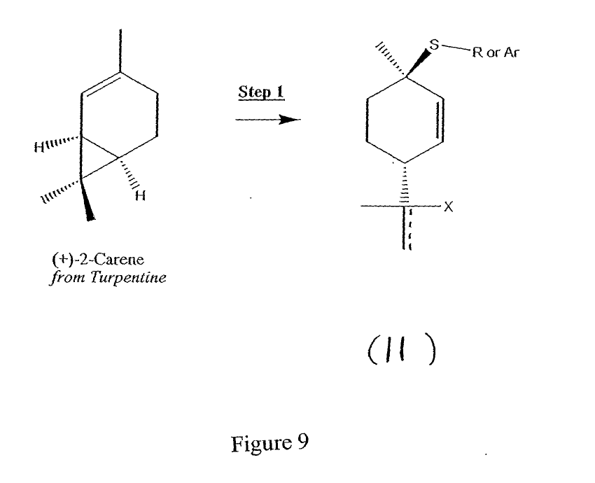

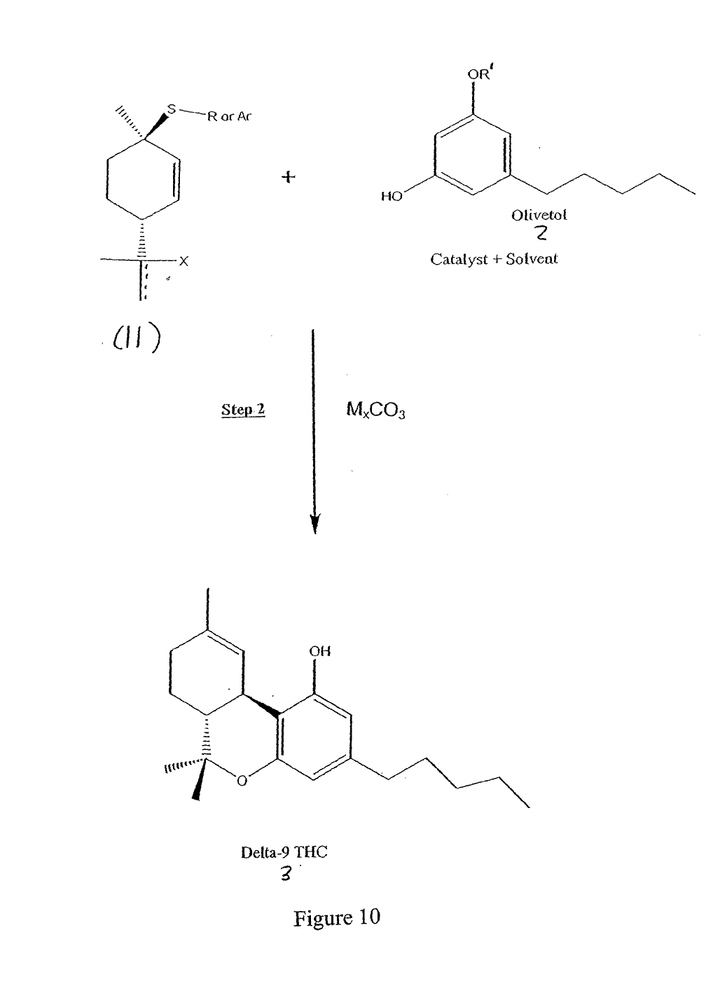

In a yet another version of the invention, an intermediate II according to the invention is first synthesized and then reacted with olivetol to produce Delta-9 tetrahydrocannabinol 3 as shown in FIGS. 9 and 10. Referring first to FIG. 9, (+)-2-Carene (which is present in turpentine) is reacted according to the process described by P. B. Hopkins et al. in J. Org. Chem. 43, (1987) 1208-1217 to produce the compound II shown in FIG. 9 wherein S is sulfur or sulfoxide or sulfone; R is alkyl (e.g. methyl, ethyl, etc.) or cycloalkyl; Ar is aryl (e.g. phenyl, substituted phenyl, etc.); or another stable group; X is OH, OR, OCOR, OCOAr, O-substituted silyl groups (e.g. TMS; TRBDMS), a halogen, or nothing when the dashed line is present as a double bond with the lowermost carbon.

Turning now to FIG. 10, compound II from FIG. 9 is reacted with an unsubstituted or substituted olivetol, wherein R′ is H, alkyl, silyl, or other stable group that can be easily removed after reaction and both hydroxyls on olivetol may be suitably protected. Compound II and the olivetol are reacted in the presence of a catalyst and a solvent. Suitable catalysts include without limitation Lewis Acid catalysts such as MgX₂, ZnX₂, ScX₃, HfX₄ (X═OAc, F, Cl, Br, OTf), BF₃ complexes, BX₃ complexes where X is a halogen, metal oxides (BaO, ZnO, AgO), and metal salts. When S of compound II is sulfone or sulfoxide, the catalysts may be bases such as NaH, nBuLi, etc. Suitable solvents include without limitation dichloromethane, dichloroethane, THF, toluene, and other common solvents that do not react adversely with or destroy the catalysts. Optionally, the addition of bases such as metal carbonates (MxCO₃) may be added to remove acidic products and by-products to minimize the isomerization of delta-9 to delta-8 THC as shown in FIG. 10. Suitable bases include without limitation alkali carbonates such as Li₂CO₃, Na₂CO₃, K₂CO₃, and Cs₂CO₃. Insoluble bases are most preferred. Alkali bicarbonates (such as NaHCO₃), NaOAc, KOAc, Zn(OAc)₂, ZnO, and silica bound carbonate can also be used. Magnesium sulfate, sodium sulfate, molecular sieves, or other suitable desiccants can also be used in the presence of the Lewis acid component. This process step is also beneficial with the first version of the invention.

In one example of this version of the invention, sulfur containing compounds such as 7 shown below are converted to Δ⁹ tetrahydrocannabinol 3, or are first converted to alcohol 8 shown below which is then converted to Δ⁹ tetrahydrocannabinol 3.

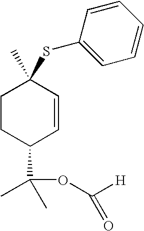

### 2-((1R,4R)-4-methyl-4-(phenylthio)cyclohex-2-enyl)propan-2-yl formate, 7

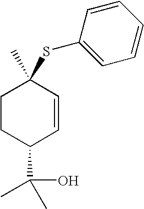

### 2-((1R,4R)-4-methyl-4-(phenylthio)cyclohex-2-enyl)propan-2-ol, 8

### EXAMPLES

The following Examples serve to illustrate the invention and are not intended to limit the invention in any way.

### Example 1

The reaction of the carbonate 6 shown in FIGS. 6 & 7 with olivetol in the presence of various Lewis acids was examined using scheme shown in FIG. 7. The results are shown in Table 1 below.

Use of BF₃-Et₂O initially gave a moderate yield of THC by HPLC with an approximately 2/1 ratio of Δ⁹-THC/Δ⁸-THC (entry 1). The addition of the inorganic K₂CO₃ led to an enhanced ratio (entry 2), while use of the organic base pyridine led to a reversal in the selectivity (entry 3). The presence of molecular sieves did not improve the ratio significantly (entry 4).

The use of BF₃-THF gave a higher ratio than BF₃-Et₂O (entry 5). The addition of K₂CO₃ to the reaction gave a synthetically useful ratio of Δ⁹-THClΔ⁸-THC (entry 6). The promising results with BF₃-THF led us to further examine this complex. Running the reaction at ambient temperature or using two equivalents of BF₃-THF (versus one equivalent) did not improve the overall yield (entries 7 and 8). Use of organic soluble hindered amine bases in combination with BF₃-THF gave no reaction by TLC analysis (entries 9 and 10). A brief solvent screen revealed that when the reaction is run in the coordinating solvents CH₃CN and THF no reaction occurs (entries 11 and 12), while use of toluene as the solvent gave the best ratio to date (entry 13). Use of other BF₃ complexes led to inferior results (entries 14-16).

Other Lewis acids were examined. Zinc bromide in the presence of molecular sieves gave a 3.9/1 ratio of Δ⁹-THC/Δ⁸-THC while ZnCl₂ led to a reversal of selectivity (entries 17 and 18). The weaker Lewis acids LiBr, MgCl₂, and Ti(O-iPr)₄ gave no Δ⁹-THC or Δ⁸-THC by HPLC (entries 19-21). It should be understood that the Lewis acids and conditions are not limited to those summarized in FIG. 7. In particular, other conditions using Lewis and Bronsted acids can potentially be used either by themselves or in the presence of other organic or inorganic bases.

TABLE 1

| | | Δ⁹-THC + | Δ⁹-THC/ |
|---|---|---|---|
| Entry | Conditions^{a} | Δ⁸-THC^{b} | Δ⁸-THC^{b} |
| 1 | BF₃-Et₂O | 71 | 1.9/1 |
| 2 | BF₃-Et₂O, K₂CO₃ | 59 | 6.3/1 |
| 3 | BF₃-Et₂O, Py, ambient temp. | 62 | 1.0/4.0 |
| 4 | BF₃-Et₂O, molecular sieves | 66 | 3.1/1 |
| 5 | BF₃-THF | 51 | 6.4/1 |
| 6 | BF₃-THF, K₂CO₃ | 40 | 12/1 |
| 7 | BF₃-THF, K₂CO₃, ambient | 40 | 3.8/1 |
| | temp. | | |
| 8 | 2 eq. BF₃-THF, K₂CO₃, 0° C.- | 56 | 1.5/1 |
| | ambient temp. | | |
| 9 | BF₃-THF, EtN(i-Pr)₂, 0° C.- | No reaction | — |
| | ambient temp. | | |
| 10 | BF₃-THF, 1,2,2,6,6- | No reaction | — |
| | pentamethylpiperidine, 0° C.- | | |
| | ambient temp. | | |
| 11 | BF₃-THF, K₂CO₃, CH₃CN, | No reaction | — |
| | 0° C.-ambient temp. | | |
| 12 | BF₃-THF, K₂CO₃, THF, 0° C.- | No reaction | — |
| | ambient temp. | | |
| 13 | BF₃-THF, K₂CO₃, PhCH₃ | 32 | 34/1 |
| 14 | BF₃-t-butyl methyl ether | 50 | 1/1.5 |
| 15 | BF₃—NH₂Et | No reaction | — |
| 16 | BF₃-Me₂S | 45 | 2.4/1 |
| 17 | ZnBr₂, molecular sieves | 58 | 3.9/1 |
| 18 | ZnCl₂, molecular sieves | 43 | 1/21 |
| 19 | LiBr, 0° C.-ambient temp. | None | — |
| 20 | MgCl₂, K₂CO₃, 0° C.-ambient | None | — |
| | temp. | | |
| 21 | Ti(O-iPr)₄, 0° C.-ambient temp. | None | — |

^{a}All reactions run in CH₂Cl₂ at 0-10° C. unless otherwise noted
^{b}By area percent HPLC

### Example 2

### Preparation of Carbonate 6 {(4aR,8aS)-4,4,7-trimethyl-4a,5,6,8a-tetrahydro-4H-benzo[d][1,3]dioxin-2-one}

To a nitrogen purged 250 mL four neck round bottom flask equipped with a magnetic stir bar, nitrogen inlet adapter, and thermometer was added 2.00 g (10.7 mmol) of diol 1 and 40 mL of pyridine. The solution was cooled to 5° C. Di-tert-butyl dicarbonate (5.84 g, 26.8 mmol) was then added followed by a 7 mL pyridine rinse. 4-(Dimethylamino)pyridine (0.31 g, 2.5 mmol) was then added at which point CO₂ evolution was evident. After stirring for 3.75 hours at 0-10° C., the reaction was allowed to warm to ambient temperature and stirred for an additional 2 hours. Saturated aqueous sodium chloride (30 mL) was then added over ca. 2 minutes at ambient temperature. Water (10 mL) was added to dissolve the solids. The quenched reaction mixture was then extracted with t-butyl-methyl ether (3×40 mL). The combined organic extracts were washed with 40 mL of water. The organic and aqueous layers were held at 0-5° C. overnight. The following morning the aqueous layer was further extracted with t-butyl-methyl ether (3×40 mL). The combined organic extracts were then dried over Na₂SO₄, and concentrated at 40° C. to give 2.17 g of the crude carbonate as a gold oil. Upon cooling to 0-5° C. the oil solidified. The solids were triturated with 7 mL of 35% ethyl acetate/heptane, filtered, washed with cold (0-5° C.) heptane (2×7 mL, 1×5 mL) and dried via high vacuum at ambient temperature to give 0.52 g of carbonate 6 as an off-white solid.

### Example 3

### Conversion of Carbonate 6 to Δ⁹-THC 3 using BF₃-Et₂O/K₂CO₃/CH₂Cl₂

To a 25 mL round bottom flask equipped with a magnetic stir bar and septa was added 35 mg (0.18 mmol) of 6, 35 mg (0.19 mmol) of olivetol, and 0.12 g (0.87 mmol) of K₂CO₃. The flask was then placed under a nitrogen atmosphere and 5 mL of CH₂Cl₂ was added. The suspension was then cooled to an external temperature of 0-10° C. BF₃-Et₂O (23 microliters, 0.18 mmol) was then added via microsyringe. The suspension gradually turned light brown. After 2 hours, 5 mL of 5% aqueous Na₂CO₃ was added to the reaction. The reaction was stirred for 15 minutes, the layers were separated and the organic layer was dried over Na₂SO₄. The dried organic layer was then purged with nitrogen to remove the majority of the solvent. Area percent HPLC analysis of the resulting oil indicated a 6.3/1 ratio of Δ⁹-THC/Δ⁸-THC.

### Example 4

### Conversion of Carbonate 6 to Δ⁹-THC using BF₃-THF/K₂CO₃/CH₂Cl₂

To a 25 mL round bottom flask equipped with a magnetic stir bar and septa was added 50 mg (0.25 mmol) of carbonate 6, 51 mg (0.28 mmol) of olivetol, and 0.14 g (1.0 mmol) of K₂CO₃. The flask was then placed under a nitrogen atmosphere and 5 mL of CH₂Cl₂ was added. The suspension was then cooled to an external temperature of 0-10° C. BF₃-THF (29 microliters, 0.26 mmol) was then added via microsyringe. The slurry turned light yellow during the addition. After 3.25 hours, 5 mL of 5% aqueous Na₂CO₃ was added to the reaction. The layers were separated and the organic layer was dried over Na₂SO₄. The dried organic layer was then purged with nitrogen to remove the majority of the solvent. Area percent HPLC analysis of the resulting oil indicated a 12/1 ratio of Δ⁹-THC/Δ⁸-THC.

### Example 5

### Conversion of Carbonate 6 to Δ⁹-THC using BF₃-THF/K₂CO₃/PhCH₃

To a 25 mL round bottom flask equipped with a magnetic stir bar and septa was added 50 mg (0.25 mmol) of carbonate 6, 52 mg (0.29 mmol) of olivetol, and 0.14 g (1.0 mmol) of K₂CO₃. The flask was then placed under a nitrogen atmosphere and 5 mL of PhCH₃ was added. The suspension was then cooled to an external temperature of 0-10° C. BF₃-THF (29 microliters, 0.26 mmol) was then added via microsyringe. The slurry turned light yellow during the addition. After 2 hours, 5 mL of 5% aqueous Na₂CO₃ was added to the reaction. The layers were separated and the organic layer was dried over Na₂SO₄. Area percent HPLC analysis of the resulting solution indicated a 34/1 ratio of Δ⁹-THC/Δ⁸-THC.

### Example 6

### Preparation of Phenylsulfenyl Chloride

To a nitrogen purged 50 mL three-necked round bottom flask equipped with a magnetic stirring bar, nitrogen inlet, water-cooled condenser with a nitrogen outlet adapter, and thermocouple, was added 3.40 g (25.5 mmol) of N-Chlorosuccinimide and 25 mL of dichloromethane. The solution was air cooled while 0.57 mL thiophenol was added, and an immediate orange color and exotherm were noted. With good stirring more thiophenol was added over about 10 minutes. The reaction boiled gently during the addition of a total of 2.807 g (25.5 mmol) thiophenol, then stirred for an additional 20 minutes to cool to room temperature. Solid succinimide precipitated during most runs from this 1.0 M Phenylsulfenyl chloride solution. Such a solution can be stored at room temperature under nitrogen if protected from light. This procedure follows the preparation and use of this reagent for other substrates by Paul B. Hopkins and Philip L. Fuchs: J. Org. Chem. 43, 1208-1217 (1987).

### Example 7

### Preparation of Phenylthio Formate 7

To a nitrogen purged 50 mL three-necked round bottom flask equipped with a magnetic stirring bar, nitrogen inlet and outlet adapters, and thermocouple, was added 1.00 mL (+)-2-Carene (6.33 mmol) and 25 mL DMF. The solution was cooled to −55°<T<−50′, while 6.3 mL 1.0 M phenylsulfenyl chloride solution in dichloromethane was added over about 10 minutes. The color of the reagent solution immediately dissipated as it hit the DMF solution. After a few minutes the reaction was allowed to warm to above zero, and poured into a mixture of 50 mL EtOAc and 50 mL saturated Na₂CO₃ solution. More water was added, and lower aqueous layer had a pH of about 10. The layers were separated, the organic layer was washed with water, dried over MgSO₄, and stripped to 1.53 g yellow oil. TLC (10% EtOAc-Hex) showed UV+ spots that stained with 5% PMA at: 0.07 (phenylthio alcohol, 8); 0.34 (phenylthio formate, 7); 0.56 (diphenyl disulfide); and less polar material that included an elimination product, 9:

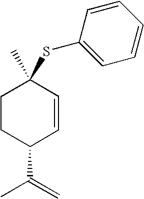

### ((1R,4R)-1-methyl-4-(prop-1-en-2-yl)cyclohex-2-enyl)(phenyl)sulfane, 9

Such a mixture can be purified by column or flash chromatography on silica gel with 2-5% MtBE-Heptane, yielding 30-35% formate ester 7. IR, HMR, CMR, and HPLC-UV spectra confirmed the structure. Pure formate ester or a mixture of formate ester and alcohol can be converted to the alcohol 8, as shown next.

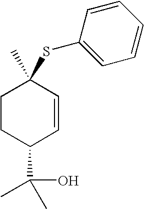

### 2-((1R,4R)-4-methyl-4-(phenylthio)cyclohex-2-enyl)propan-2-ol, 8

### Example 9

### Conversion of Phenylthio Formate 7 to Phenylthio Alcohol 8

2.20 g formate 7+ alcohol 8 mixture was dissolved in 20 mL methanol, to which 1.0 mL 25% NaOMe in methanol was added. After stirring for 2 minutes at room temperature, TLC showed complete conversion to the alcohol, 8. The reaction was poured into a separatory funnel along with 50 mL EtOAc and 50 mL 10% HOAc/water. The pH of the aqueous layer was 4. The layers were separated; the organic layer was washed with water and dried over MgSO₄, and stripped to 1.90 g waxy white solid. Purification by column chromatography provided 920 mg 8, 90.6% pure by HPLC, with diphenyl disulfide and the elimination product, 9, as the impurities. This yield represented a 37.5% yield based the 90.6% purity, and on starting with 8.48 mmoles phenylsulfenyl chloride and 9.5 mmoles (+)-2-carene.

### Example 10

### Conversion of Alcohol 8 to Δ⁹-THC using BF₃-Et₂O/Na₂CO₃/CH₂Cl₂

To a 25 mL round bottom flask equipped with a magnetic stir bar and septa was added 260 mg (1.00 mmol) 8, 193 mg (1.07 mmol) of olivetol, and about 150 mg Na₂CO₃, under a nitrogen atmosphere with 10 mL of CH₂Cl₂. The suspension was then cooled to an external temperature of −20° C. BF₃-Et₂O (3×40 uL: 0.95 mmol) was then added via syringe. The suspension showed an immediate light yellow color. After 20 minutes, the temperature was −11° C. There was Δ⁹-THC present, along with starting material and thiophenol, but no Δ⁸-THC was detected. After warming to zero, the area percent by HPLC analysis of the resulting reaction mixture was 93 to 7, Δ⁹-THC to Δ⁸-THC.

### Example 11

### Preparation of 2-carene epoxide from 2-Carene

To a 500 mL 4 neck round bottom flask equipped with a magnetic stir bar and nitrogen inlet adapter was added 12.43 g (147.9 mmol) of NaHCO₃ and 75 mL of distilled water. To the suspension was added 150 mL of methylene chloride followed by 15.00 g (110.3 mmol) of 2-carene. To the biphasic mixture was added 24.32 g (108.5 mmol) of 3-chloroperbenzoic acid (MCPBA) portionwise over 1.5 hours at an internal temperature of 17-24° C. After stirring for an additional 30 minutes the reaction was determined to be complete by thin layer chromatography. The layers were separated and the aqueous layer was extracted with methylene chloride (2 times 75 mL). The combined organic layers were washed with saturated NaHCO₃ (2 times 75 mL) followed by water (100 mL). The washed organic layer was dried over Na₂SO₄, concentrated, and dried via high vacuum to give 15.92 g of 2-carene epoxide as an oil (95% yield).

### Example 12

### Preparation of (+)-p-Menth-2-ene-1,8-diol 1 from 2-carene epoxide 5a

To a 25 mL round bottom flask equipped with a magnetic stir bar and septum was added 0.25 g ((1.6 mmol) of 2-carene epoxide and 2.5 mL of heptane. The solution was placed under nitrogen and cooled to an external temperature of ca. 0-10° C. Water (0.05 mL, 3 mmol) was added followed by 1 drop of acetic acid. After 2 hours, the reaction was determined to be complete by thin layer chromatography. To the thick slurry was added an additional 8 mL of heptane to aid in the filtration. The slurry was then filtered, washed with 0-5° C. heptane (3 times 3 mL), and dried in a vacuum oven at 40° C. to give 0.22 g of diol 1 as a white solid (79% yield).

### Example 13

### Preparation of a 2-carene epoxide and 3-carene epoxide Mixture

To a 2 liter 4 neck round bottom flask equipped with a mechanical stirrer, nitrogen inlet adapter and temperature probe was charged 46.0 g (338 mmol) of a 43/57 mixture (by GC) of 2-carene/3-carene, 460 mL of methylene chloride, and a suspension of 38.2 g (455 mmol) of NaHCO₃ in 270 mL of distilled water. To the biphasic mixture at an internal temperature of 23° C. was added 75.0 g (335 mmol) of 3-chloroperbenzoic acid (MCPBA) portionwise over 1.75 hours at an internal temperature of 23-32° C. The reaction was stirred for an additional 20 minutes after the completion of the addition at which point the reaction was complete as determined by thin layer chromatography. The layers were then separated and the aqueous layer was extracted with 300 mL of methylene chloride. The combined organic extracts were washed with 250 mL of saturated NaHCO₃ followed by 300 mL of water. The solution containing the epoxide mixture was then dried over Na₂SO₄ and concentrated to give 47.3 g of 2-carene epoxide and 3-carene epoxide (92% yield).

### Example 14

### Preparation of (+)-p-Menth-2-ene-1,8-diol 1 from a 2-carene epoxide and 3-carene epoxide Mixture

To a 1 liter 4 neck round bottom flask equipped with a mechanical stirrer, nitrogen inlet adapter and temperature probe was charged 45.0 g of the 2-carene epoxide and 3-carene epoxide mixture of Example 13 (127 mmol of contained 2-carene epoxide), 450 mL of heptane and 10.1 mL (561 mmol) of distilled water. The biphasic mixture was cooled to an internal temperature of 0-10° C. Acetic acid (1.5 mL, 26 mmol) was then added and the mixture was stirred vigorously at 0-10° C. After 3 hours an additional 5.0 mL (280 mmol) of distilled water was added. After 4.5 hours the reaction was determined to be complete by thin layer chromatography. The resulting slurry was filtered, washed with 0-5° C. heptane (3 times 80 mL) and dried at 40° C. overnight in a vacuum oven to give 13.9 g of (+)-p-Menth-2-ene-1,8-diol 1 as a white solid (64% yield based on contained 2-carene epoxide in starting mixture). The amorphous product can be recrystallized from ethyl acetate (4 mL/g) and heptane (8 mL/g) to give (+)-p-Menth-2-ene-1,8-diol 1 as a crystalline solid (78% recovery).

### Example 15

### Preparation of (+)-p-Menth-2-ene-1,8-diol using Acetic Acid in Various Solvents

The mixture of 2-carene epoxide and 3-carene epoxide was treated with water (ca. 3.0 eq.) and catalytic acetic acid (0.1 eq.) in various solvents (ca. 9 mL/g) at 0-5° C. The results are shown below in Table 2.

TABLE 2
Preparation of (+)-p-Menth-2-ene-1,8-diol in Various Solvents

| Entry | Solvent | Percent Yield |
|---|---|---|
| 1 | Heptanes | 87 |
| 2 | Cyclohexane | 53 |
| 3 | Toluene | 65 |
| 4 | CHCl₃ | 29 |
| 5 | Methyl-t-butyl-ether (MTBE) | 68 |
| 6 | MTBE:Heptanes (1:3) | 33 |
| 7 | CH₂Cl₂ | 52 |
| 8 | CH₂Cl₂:Heptanes (1:1) | 48 |
| 9 | Isopropyl acetate/Heptanes | 61 |
| | (1:10) | |
| 10 | t-Butanol/Heptanes (1:50) | 54 |

### Example 16

### Preparation of (+)-p-Menth-2-ene-1,8-diol 1 using Various Acid Catalysts in Heptane

The mixture of 2-carene epoxide and 3-carene epoxide was treated with water (ca. 3.0 eq.) and an acid catalyst (0.1 eq.) in heptanes (ca. 9 mL/g) at 0-5° C. The results are shown below in Table 3.

TABLE 3
Preparation of (+)-p-Menth-2-ene-1,8-diol
1 using Various Acid Catalysts

| Entry | Acid Catalyst | Percent Yield |
|---|---|---|
| 1 | Acetic acid and t-butanol (0.2 eq.) | 54 |
| 2 | Benzoic acid | 71 |
| 3 | Formic acid | 66 |
| 4 | Trifluoroacetic acid | 18 |
| 5 | KH₂PO₄ | 26 |

Thus, the present invention provides processes for the synthesis of Delta-9 tetrahydrocannabinol which result in an improved Δ⁹-THC/Δ⁸-THC ratio, and intermediates that may be used in the synthesis of Delta-9 tetrahydrocannabinol such that improved Δ⁹-THC/Δ⁸-THC ratios are achieved.

The present invention also provides a scaleable process for the preparation of (+)-p-menth-2-ene-1,8-diol, an intermediate used in the synthesis of delta-9-tetrahydrocannibinol.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

### INDUSTRIAL APPLICABILITY

The present invention relates to methods and intermediates for the synthesis of Delta-9 tetrahydrocannabinol, a tricyclic terpene currently being used for appetite stimulation in cancer and AIDS patients.

What is claimed is:

1. A process for producing (+)-p-Menth-2-ene-1,8-diol, the process comprising:
(a) preparing a reaction mixture including a solvent in which (+)-p-Menth-2-ene-1,8-diol is insoluble, 2-carene epoxide, water, and an acid catalyst such that (+)-p-Menth-2-ene-1,8-diol precipitates from the reaction mixture; and
(b) filtering the reaction mixture to remove (+)-p-Menth-2-ene-1,8-diol from the reaction mixture.

2. The process of claim 1 wherein:
the solvent is a non-nucleophilic organic solvent.

3. The process of claim 1 wherein:
the solvent is a C₅-C₁₂ alkane.

4. The process of claim 1 wherein:
the solvent is heptane.

5. The process of claim 1 wherein step (a) comprises:
mixing the solvent and 2-carene epoxide,
adjusting the temperature of the reaction mixture to 15° C. or below, and
thereafter adding the water and the acid catalyst.

6. The process of claim 1 wherein step (a) comprises:
mixing the solvent and a mixture of 2-carene epoxide and 3-carene epoxide,
adjusting the temperature of the reaction mixture to 15° C. or below, and
thereafter adding the water and the acid catalyst.

7. The process of claim 1 wherein:
the catalyst is selected from the group consisting of aliphatic carboxylic acids, aromatic carboxylic acids, sulfonic acids, pyridinium acids, ammonium acids, and mixtures thereof.

8. The process of claim 1 wherein:
the catalyst is soluble in the solvent.

9. The process of claim 1 wherein:
the catalyst is acetic acid.

10. The process of claim 1 wherein:
(+)-p-Menth-2-ene-1,8-diol is produced in a yield of at least 70%.

11. A process for producing (+)-p-Menth-2-ene-1,8-diol, the process comprising:
(a) preparing a reaction mixture including a C₅-C₁₂ alkane solvent in which (+)-p-Menth-2-ene-1,8-diol is insoluble, 2-carene epoxide, water, and acetic acid such that (+)-p-Menth-2-ene-1,8-diol precipitates from the reaction mixture; and
(b) filtering the reaction mixture to remove (+)-p-Menth-2-ene-1,8-diol from the reaction mixture.

12. The process of claim 11 wherein:
the solvent is heptane.

13. The process of claim 12 wherein step (a) comprises:
mixing the heptane and 2-carene epoxide,
adjusting the temperature of the reaction mixture to 15° C. or below, and
thereafter adding the water and the acetic acid.

14. The process of claim 12 wherein step (a) comprises:
mixing the solvent and a mixture of 2-carene epoxide and 3-carene epoxide,
adjusting the temperature of the reaction mixture to 15° C. or below, and
thereafter adding the water and the acetic acid.

15. The process of claim 12 wherein:
the solvent is heptane.

16. The process of claim 15 wherein:
the (+)-p-Menth-2-ene-1,8-diol is recrystallized after filtering.

17. A method for preparing Delta-9 tetrahydrocannabinol, the method comprising:
reacting a compound of the formula 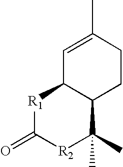
wherein R₁ is selected from O, N and S, and R₂ is selected from O, N and S, with unsubstituted resorcinol or a substituted resorcinol.

18. The method of claim 17 wherein:
the compound has the formula

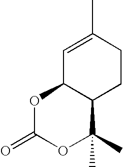

19. The method of claim 17 wherein:
the compound is reacted with olivetol.

20. The method of claim 17 wherein:
the compound is reacted with olivetol in the presence of an acid.

21. The method of claim 20 wherein:
the acid is selected from Lewis acids, Bronsted acids, and mixtures thereof.

22. The method of claim 20 wherein:
the acid is selected from Bronsted acids and mixtures thereof.

23. The method of claim 17 wherein:
the compound is reacted with olivetol in the presence of an acid and a material selected from metal sulfates, molecular sieves, and desiccants.

24. The method of claim 17 wherein:
the compound is reacted with olivetol in the presence of an inorganic or organic base.

25. The method of claim 24 wherein:
the base is an alkali metal carbonate, an alkali metal bicarbonate, a metal acetate, a metal oxide, or silica bound carbonate.

26. The method of claim 17 wherein:
the compound is a chiral, non-racemic substance, and
the Delta-9 tetrahydrocannabinol is a non-racemic substance.

27. A method for preparing Delta-9 tetrahydrocannabinol, the method comprising:
reacting a compound of the formula 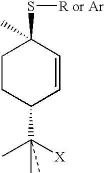
wherein S is sulfur or sulfoxide or sulfone; R is alkyl or cycloalkyl; Ar is aryl; and X is OH, OR, OCOR, OCOAr, O-substituted silyl groups, a halogen, or nothing when the dashed line is present as a double bond with the lowermost carbon, with unsubstituted resorcinol or a substituted resorcinol.

28. The method of claim 27 wherein:
the compound is reacted with olivetol.

29. The method of claim 27 wherein:
the compound is reacted with olivetol in the presence of an acid.

30. The method of claim 29 wherein:
the acid is selected from Lewis acids, Bronsted acids, and mixtures thereof.

31. The method of claim 29 wherein:
the acid is selected from Bronsted acids and mixtures thereof.

32. The method of claim 27 wherein:
the compound is reacted with olivetol in the presence of an acid and a material selected from metal sulfates, molecular sieves, and desiccants.

33. The method of claim 27 wherein:
the compound is reacted with olivetol in the presence of an inorganic or organic base.

34. The method of claim 33 wherein:
the base is an alkali metal carbonate, an alkali metal bicarbonate, a metal acetate, a metal oxide or silica bound carbonate.

35. The method of claim 27 wherein:
the compound is a chiral, non-racemic substance, and the Delta-9 tetrahydrocannabinol is a non-racemic substance.

36. A method for preparing Delta-9 tetrahydrocannabinol, the method comprising:
reacting unsubstituted or substituted olivetol with a cyclic compound in the presence of an acid and a base.

37. The method of claim 36 wherein:
the base is insoluble in the reaction medium.

38. The method of claim 36 wherein the cyclic compound has the following formula: 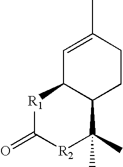
wherein R₁ is selected from O, N and S, and R₂ is selected from O, N and S.

30. The method of claim 36 wherein the cyclic compound has the following formula:

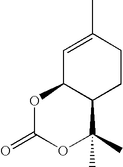

40. The method of claim 36 wherein the cyclic compound has the following formula: 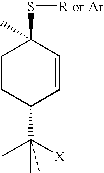
wherein S is sulfur or sulfoxide or sulfone; R is alkyl or cycloalkyl; Ar is aryl; and X is OH, OR, OCOR, OCOAr, O-substituted silyl groups, a halogen, or nothing when the dashed line is present as a double bond with the lowermost carbon.

41. The method of claim 36 wherein:
the base is an alkali metal carbonate, an alkali metal bicarbonate, a metal acetate, a metal oxide or silica bound carbonate.

42. The method of claim 36 wherein:
the acid is selected from Lewis acids, Bronsted acids, and mixtures thereof.

43. The method of claim 42 wherein:
the acid is selected from Bronsted acids and mixtures thereof.
